(19) United States
(12) Patent Application Publication
(10) Pub. No.: US 2021/0259931 A1
(43) Pub. Date: Aug. 26, 2021

(54) DENTIFRICE COMPRISING CARBOXYLIC ACID OR ALKALI METAL SALT THEREOF AND A SOURCE OF FREE FLUORIDE IONS

(71) Applicant: GlaxoSmithKline Consumer Healthcare (UK) IP Limited, Brentford, Middlesex (GB)

(72) Inventors: Jonathan Edward CREETH, Surrey (GB); Shazada Yassar KHAN, Surrey (GB); Richard LYNCH, Surrey (GB); David URQUHART, Surrey (GB)

(21) Appl. No.: 17/252,372

(22) PCT Filed: Jul. 3, 2019
(86) PCT No.: PCT/EP2019/067791

§ 371 Date: Dec. 15, 2020

(30) Foreign Application Priority Data
Jul. 5, 2018 (GB) 1811061.9

(51) Int. Cl. A61K 8/21; A61K 8/365; A61K 8/81; A61Q 11/00

(52) U.S. Cl. CPC A61K 8/21; A61K 8/365; A61K 2800/92; A61Q 11/00; A61K 2800/591; A61K 8/8164

(57) ABSTRACT

A dentifrice composition is described comprising a carboxylic acid or alkai metal salt thereof, a source of free fluoride ions and optionally a copolymer of methyl vinyl ether with maleic anhydride or acid. Importantly the dentifrice composition is mildly acidic, having a slurry pH in the range greater than 5.0 to less than 6.5. The composition enhances fluoride uptake into teeth and provides protection against acidic challenges.

### FIELD OF THE INVENTION

The present invention relates to a dentifrice composition for strengthening and protecting enamel of natural teeth, thereby providing protection against acidic challenges. A composition according to the invention comprises a particular carboxylic acid or alkali metal salt thereof, a source of free fluoride ions and optionally a copolymer of methyl vinyl ether (MVE) with maleic anhydride or acid. Importantly the dentifrice composition is mildly acidic, having a slurry pH in the range greater than 5.0 to less than 6.5.

### BACKGROUND OF THE INVENTION

Tooth mineral is composed predominantly of calcium hydroxyapatite, Ca₁₀(PO₄)₆(OH)₂, which may be partially substituted with anions such as carbonate or fluoride, and cations such as zinc or magnesium. Tooth mineral may also contain non-apatitic mineral phases such as octacalcium phosphate and calcium carbonate.

Tooth decay may occur as a result of dental caries, which is a multifactorial disease where bacterial acids such as lactic acid produced by metabolism of dietary sugars leads to sub-surface demineralisation that does not fully remineralise in between sugar exposures, resulting in progressive tissue loss and eventually cavity formation. The presence of a plaque biofilm is a prerequisite for dental caries, and acidogenic bacteria such as Streptococcus mutans may become pathogenic when levels of sugars (i.e. easily fermentable carbohydrate such as sucrose), are elevated for extended periods of time.

Even in the absence of a plaque biofilm, loss of dental hard tissues can occur as a result of acid erosion and/or physical tooth wear; these processes are believed to act synergistically. Exposure of the dental hard tissues to acid can cause demineralisation, resulting in surface softening and a decrease in mineral density. This softened mineral is vulnerable to wear from physical contact. Under normal physiological conditions, partially demineralised tissues self-repair through the remineralising effects of saliva. Saliva is supersaturated with respect to calcium and phosphate, and in healthy individuals, saliva secretion serves to wash out the acid challenge, and to raise the pH so as to alter the equilibrium in favour of mineral deposition.

Dental erosion (i.e. acid erosion or acid wear) is a surface phenomenon that involves demineralisation, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinaigrette. Dental erosion may also be caused by repeated contact with hydrochloric acid (HCI) produced by the stomach, which may enter the oral cavity through an involuntary response such as gastroesophageal reflux, or through an induced response as may be encountered in sufferers of bulimia.

Tooth wear (i.e. physical tooth wear) is caused by attrition and/or abrasion. Attrition occurs when tooth surfaces rub against each other, a form of two-body wear. An often-dramatic example is that observed in subjects with bruxism, a tooth-grinding habit during sleep where the applied forces are high, and is characterised by accelerated wear, particularly on the occlusal surfaces. Abrasion typically occurs as a result of three-body wear, and the most common example is that associated with brushing with a toothpaste. In the case of fully mineralised enamel, levels of wear caused by commercially available toothpastes are minimal and of little or no clinical consequence. However, if enamel has been demineralised and softened by exposure to an erosive challenge, the enamel becomes more susceptible to wear. Enamel is thinnest at its junction with the dentine, which in health is located just below the gum margin. However, gum recession (especially associated with ageing) can expose the enamel-dentine junction and wear of enamel in this region can expose dentine, leading to hypersensitivity, as described below.

Dentine is a vital tissue that in vivo is normally covered by enamel or cementum depending on the location i.e. crown versus root respectively. Dentine has a much higher organic content than enamel and its structure is characterised by the presence of fluid-filled tubules that run from the surface of the dentine-enamel or dentine-cementum junction to the pulp interface. Dentine is much softer than enamel and consequently is more susceptible to wear. Subjects with exposed dentine should avoid the use of highly abrasive toothpastes. Again, softening of dentine by an erosive challenge will increase susceptibility of the tissue to wear. It is widely accepted that the origins of dentine hypersensitivity relate to changes in fluid flow in exposed tubules, (the hydrodynamic theory), that result in stimulation of mechanoreceptors thought to be located close to the pulp interface. Not all exposed dentine is sensitive since it is generally covered with a smear layer; an occlusive mixture comprised predominantly of mineral and proteins derived from dentine itself, but also containing organic components from saliva. Over time, the lumen of the tubule may become completely occluded with mineralised tissue. The formation of reparative dentine in response to trauma or chemical irritation of the pulp is also well-documented. Nonetheless, an erosive challenge can remove the smear layer and tubule “plugs” releasing dentinal fluid flow, making the dentine much more susceptible to external stimuli such as hot, cold and pressure. As previously indicated, an erosive challenge can also make the dentine surface much more susceptible to wear. In addition, dentine hypersensitivity worsens as the diameter of the exposed tubules increases, and since the tubule diameter increases as one proceeds in the direction of the pulp interface, progressive dentine wear can result in an increase in hypersensitivity, especially in cases where dentine wear is rapid.

Erosion and/or acid-mediated tooth wear are therefore primary aetiological factors in the development of dentine hypersensitivity.

It has been claimed that an increased intake of dietary acids, and a move away from formalised meal times, has been accompanied by a rise in the incidence of dental erosion and tooth wear in the populations of developed countries. In view of this, oral care compositions which can help prevent dental erosion and tooth wear and which provide protection from dental caries would be advantageous.

Oral care compositions often contain a source of fluoride ions for promoting remineralisation of teeth and for increasing the acid resistance of dental hard tissues. To be effective the fluoride ions must be available for uptake into the dental hard tissues being treated.

It has been observed that demineralised enamel will take up more fluoride from an acidic solution than from a neutral one (e.g. Friberger, The effect of pH upon fluoride uptake in intact enamel. Scand. J. Dent. Res. (1975) 83:339-344). The Friberger study investigated the in vitro uptake of fluoride from dentifrice slurries and from sodium fluoride solutions of different pH ranging from 7.1 to 4.5. The pH was adjusted with a few drops of 0.1 M HCl acid or NaOH. The investigation showed there was no significant difference between the agents (namely a sodium fluoride dentifrice, a potassium fluoride and manganese chloride dentifrice and a sodium fluoride solution of the same fluoride concentration), but that the effect of pH was significant. The uptake of fluoride in the form of fluorapatite was more than five times larger at the lower pH level.

GB 1,018,665 (Unilever Ltd) describes a fluoride dentifrice incorporating a water-soluble buffering system that comprise a weak organic acid and an alkali metal salt, for example acetic acid/sodium acetate and malic acid/sodium malate, and wherein the pH of a slurry of the dentifrice in simulated saliva is from 5 to 6. The dentifrice is disclosed as being capable of reducing enamel solubility compared to solutions at neutral pH.

US 2009/0087391A1 (Joziak) describes a foaming fluoride dental composition comprising a surface-active agent selected from the group consisting of non-ionic, zwitterionic or betaine surfactants or mixtures thereof and an acidifying agent in an amount sufficient to adjust the pH to 3 to 5. Suitable acidifying agents are organic acids such as malic acid, hydrosuccinic acid, citric acid and tartaric acids or mixtures thereof.

WO 01/66074 (Colgate) describes a dual-component dentifrice, one phase being alkaline and containing fluoride ions, the other phase being acidic and containing phosphate ions, which on mixing prior to use, provides an acidic phosphate fluoride composition (pH 4 to 6). It is suggested that the delivery of the dentifrice at an acidic pH can enhance the uptake of the fluoride ions into the tooth enamel.

U.S. Pat. No. 4,363,794 (Lion Corporation) discloses an oral composition which comprises a stannous salt such as stannous fluoride, a water-soluble fluoride salt such as sodium fluoride and an orally acceptable acid such as L-ascorbic acid, lactic acid, malonic acid, tartaric acid, citric acid, hydrochloric acid and pyrophosphoric acid, the molar ratio of fluoride ion to stannous ion being in the range of 3.2 to 7:1, preferably 3.5 to 6:1, in an aqueous condition and the pH of the composition being in the range of from 2 to 4. The composition is disclosed as exhibiting excellent effects on the inhibition of dental caries. According to U.S. Pat. No. 4, 363,794, the specified pH range results in an increase of effectiveness on the increment of acid-resistance for the treated enamel and on stability of the stannous ion. Low pH (below 2) tends to pose an obstacle to oral application of the composition whereas a pH above 4 often causes reduced availability and stability of stannous ion.

The use of fluoride-containing dentifrices formulated at substantially neutral pH, have also been described in the art for remineralizing and strengthening teeth. WO2006/1000071 (Glaxo Group Ltd) discloses dentifrice compositions that comprise, amongst other ingredients, a fluoride ion source, and have a pH in the range 6.5 to 7.5. Such compositions have been commercialized as SENSODYNE Pronamel toothpaste for use in protecting teeth against dietary acidic challenges.

In one aspect the present invention is based on the discovery that incorporation of a particular carboxylic acid(s) as described herein in a mildly acidic dentifrice composition comprising a source of fluoride ions, advantageously enhances the uptake of fluoride ions into dental enamel when compared to the same composition under a neutral pH, or when compared to the same mildly acidic composition but containing a different carboxylic acid (such as malic acid), or an inorganic acid (such as phosphoric acid).

In a further aspect the present invention is based on the discovery that incorporation of a copolymer of methyl vinyl ether with maleic anhydride or acid provides a further benefit of significantly increasing enamel solubility reduction without adversely impacting on uptake of fluoride.

The use of copolymers based on methyl vinyl ether and maleic acid in oral care compositions is known in the art. For example, U.S. Pat. No. 4,485,090 discloses dentifrice compositions comprising a polymeric anionic membrane-forming material such as “Gantrez AN”. According to U.S. Pat. No. 4,485,090 the material attaches itself to tooth surfaces and forms a substantially continuous barrier thereon by complexing with calcium present in the teeth. The barrier formed is described as substantially reducing elution of a previously applied therapeutic agent (e.g. dental fluoride treatment), thereby prolonging the effectiveness of such agent. According to U.S. Pat. No. 4,485,090, compositions of the invention therein need only be applied periodically (e.g. once daily) in order to achieve the desired reduction in elution and resultant control of caries and plaque.

Later-filed US patent application US2004/0146466 (Baig et al) discloses that particular polymeric mineral surface active agents such as synthetic anionic polymers e.g. polyacrylates and copolymers of maleic anhydride or acid and methyl vinyl ether (e.g. Gantrez), have a strong affinity for tooth enamel surface and that such polymers deposit a layer or coating on the enamel surface. Effective amounts of a polymeric mineral surface active agent are described as ranging from about 1% to about 35%, preferably from about 2 to about 30%, more preferably from about 5% to about 25%, and most preferably from about 6% to about 20% by weight of the total oral composition.

WO2007/069429 (Lion Corporation) discloses toothpaste compositions containing (A) from 0.3 to 1.2% by mass of at least one linear and water-soluble polyphosphate represented by the general formula Mₙ₊₂PₙO₃ₙ₊₁ (wherein M represents Na or K; and n is an integer of 2 or 3), (B) from 0.1% to 2.0% by mass of a methyl vinyl ether/maleic anhydride copolymer, a 2.0% by mass aqueous solution of which has a viscosity of from 5 to 1000 mPa·s at 25° C. and pH 7.0, (C) from 0.6 to 2.0% by mass of a lauryl sulphate, and (D) from 0.2 to 1.0% by mass of a betaine type amphoteric surfactant, and the composition ratio by mass (C)/(D) ranging from 1 to 4. Such compositions are described as causing low irritation of the oral mucosa and as providing favourable foaming in use, as well as having an excellent effect on preventing the adhesion of stains to tooth surfaces.

WO2011/094499 (Colgate-Palmolive Company) discloses anti-erosion oral care formulations comprising a copolymer of a methyl vinyl ether and a maleic anhydride, such as Gantrez, and a metal compound or salt that becomes more soluble at acidic pH. According to W02011/094499, a mucoadhesive polymer, such as Gantrez, may be incorporated into the orally acceptable vehicle in an amount ranging from 0.01 to 20% by weight, preferably 0.1 to 10% by weight and most preferably from 0.5 to 7% by weight of component. A “Low Polymer Formulation” and a “High Polymer Formulation” exemplified in WO2011/094499 comprises, respectively 0.5% and 2.0% by weight Gantrez.

A Technical Information Sheet, Bulletin VC-862A, published by Ashland Speciality Chemicals (Rev. 02-2015), reported that superior acid erosion resistance of enamel had been observed in an in vitro study, following pre-treatment of the enamel with a toothpaste containing 2% Gantrez S-97 polymer, and that the presence of the Gantrez was believed to be the primary reason for the improvement observed in reducing acid erosion.

WO2015/171836 (Procter & Gamble) describes oral care compositions containing 5% metal ions, at least 0.001% of stannous ions and optionally from about 0.001% to about 4% zinc ions; at least about 100 ppm by weight of fluoride ions and at least about 0.03% by weight of a mineral surface active agent selected from, amongst others, copolymers of maleic anhydride or acid with methyl vinyl ether; at least 5% water; less than 10% by weight of a fused silica, calcium based abrasive and mixtures thereof, less than 5% of polyphosphates having n+3 or higher, wherein the weight ratio of total metal ion (stannous optionally zinc) is equal to or less than 0.5. WO2015/171836 discloses by appropriately balancing the ratio of total metal ions to a selected group of mineral surface active agents that fluoride uptake may be improved and specified benefits (antibacterial efficacy, fluoride uptake, demineralization and reduced stain) necessary to hit a “sweet spot” of oral care can be achieved in one composition. According to WO2015/171836, the compositions described therein provide remineralization enhancement and demineralization inhibition benefits by controlling deposition of surface protection agents, which when deposited in excess negatively impact fluoride uptake and remineralization of tooth lesions below the surface. The inclusion of a buffering agent is optional and the oral compositions will typically have a pH from about 4 to about 7, preferably from about 4.5 to about 6.5 and more preferably from about 5 to about 6. W02015/171836 discloses that the inclusion of Gantrez does not impact fluoride uptake from a NaF containing formula.

### SUMMARY OF THE INVENTION

In one aspect the present invention provides a dentifrice composition comprising a carboxylic acid or alkali metal salt thereof wherein the acid is selected from the group consisting of malonic acid, glutaric acid, tartaric acid, lactic acid and mixtures thereof; and a source of free fluoride ions; and wherein the composition has a slurry pH in the range from greater than 5.0 to less than 6.5.

In a further aspect the invention provides a dentifrice composition comprising a carboxylic acid or alkali metal salt thereof wherein the acid is selected from the group consisting of malonic acid, glutaric acid, tartaric acid, lactic acid and mixtures thereof; a source of free fluoride ions; and a copolymer of methyl vinyl ether with maleic anhydride or acid; and wherein the composition has a slurry pH in the range from greater than 5.0 to less than to 6.5.

Such compositions are of use in protecting teeth against dental erosion. Such compositions are also of use in protecting teeth against dental caries.

### BRIEF DESCRIPTION OF THE DRAWINGS

FIG. 1: Effect of Malonic Acid and pH on EFU

FIG. 2: Effect of Malonic Acid and Citric Acid (at pH 5.50) on EFU

FIG. 3: Effect of Malonic Acid and pH on EFU

FIG. 4: Effect of Particular Carboxylic Acids and Phosphoric Acid on EFU

FIG. 5: Effect of Lactic Acid and pH on EFU

FIG. 6: Effect of PVM/MA (pH 6.2) on EFU

FIG. 7: Effect of PVM/MA (pH 6.2) on ESR

FIG. 8: Summary of SMHR after 4hrs Remineralization

FIG. 9: Summary of Mean %RER after 4hrs Remineralization

FIG. 10: Summary of EFU 4hrs Remineralization

FIG. 11: Tissue loss data after treatment of human enamel with dentifrices followed by an erosive challenge

FIG. 12: Variation of Mean Fluoride Uptake over 50 μm depth

FIG. 13: Mean relative 44 Ca uptake over 20 μm depth

### DETAILED DESCRIPTION OF THE INVENTION

A composition according to the invention comprises a carboxylic acid or alkali metal salt thereof wherein the acid is selected from the group consisting of malonic acid, glutaric acid, tartaric acid, lactic acid and mixtures thereof. In one embodiment the carboxylic acid is lactic acid or an alkali metal salt thereof. Typical examples of suitable alkali metal salts include the sodium and potassium salts of the said carboxylic acids. In one embodiment the alkali metal salt is the potassium salt(s) of malonic, glutaric, tartaric, lactic acids and mixtures thereof. In one embodiment the alkali metal salt is selected from the sodium salt(s) of malonic, glutaric, tartaric, lactic acids and mixtures thereof. In one embodiment the carboxylic acid salt is potassium lactate. In one embodiment the carboxylic acid salt is sodium lactate.

The carboxylic acid or salt may be provided in the form of a solid or an aqueous solution, e.g. sodium lactate solution (60% w/w).

Suitably the carboxylic acid or alkali metal salt thereof is present in an amount of from 0.5% to 5.0% by weight of the total composition, for example from 1.0% to 4.5% or from 1.5% to 3.0% by weight of the total composition. A preferred amount is 2.0% by weight of the acid or 2.5% by weight of the salt.

A composition according to the invention comprises a source of free fluoride ions. Suitable examples of a source of free fluoride ions include an alkali metal fluoride such as sodium or potassium fluoride, polyvalent metal ion fluoride salts such as stannous fluoride, or salts of fluoride with cationic organic ions such as ammonium fluoride or bis-(hydroxyethyl) amino-propyl-N-hydroxyethyloctadecylamine-dihydrofluoride, (amine fluoride) or a mixture thereof in an amount to provide from 25 to 5000 ppm of fluoride ions, preferably from 100 to 1500 ppm. In one embodiment the source of free fluoride ions is stannous fluoride. In one embodiment the source of free fluoride ions is not stannous fluoride. In one embodiment the source of free fluoride ions is an alkali metal fluoride such as sodium fluoride. Suitably the composition contains from 0.05% to 0.5% by weight of sodium fluoride, e.g. 0.1% by weight (equating to 450 ppm of fluoride ions), 0.205% by weight (equating to 927 ppm of fluoride ions), 0.2542% by weight (equating to 1150 ppm of fluoride ions) or 0.3152% by weight (equating to 1426 ppm of fluoride ions).

A composition according to the invention is mildly acidic i.e. has a slurry pH in the range from greater than 5.0 to less than 6.5, for example from pH 5.1 to 6.4, 5.4 to 6.3, or 5.5 to 6.2. In one embodiment the pH is about 6.2. The pH referred to is that measured when the dentifrice composition is slurried with water in a 1:3 weight ratio of the composition to water. Suitably the slurry is prepared by slurring the dentifrice composition with water in a weight ratio of one part dentifrice composition and three parts distilled water. The pH is determined using a standard pH meter.

Suitably a dentifrice composition of the invention comprises a pH modifying agent to adjust the pH of the composition to the desired pH. Suitable pH modifying agents include alkali metal hydroxides such as sodium hydroxide and potassium hydroxide, or inorganic acids such as hydrochloric acid or sulphuric acid. In one embodiment the pH modifying agent is sodium hydroxide. A pH modifying agent may be used in an amount from 0.005% to 5% by weight of the composition, such as from 0.01% to 2% or 0.02% to 1% by weight of the composition.

In one aspect a composition according to the invention comprises a surface protection agent which is a copolymer of methyl vinyl ether (MVE) with maleic anhydride or acid. In one embodiment the surface protection agent is a copolymer of MVE with maleic acid. In general the copolymer is a linear copolymer comprising alternating units of MVE and maleic anhydride or acid. In one embodiment the copolymer comprises a 1:4 to 4:1 ratio of MVE : maleic anhydride or acid, such as a 1:1 ratio of MVE:maleic anhydride or acid i.e. the MVE content is about 50 mole % and the maleic anhydride or acid content is about 50 mole %. In one embodiment the copolymer is the acid form of a copolymer of MVE with maleic anhydride wherein the anhydride is fully or partially hydrolysed, e.g. following co-polymerization to provide the corresponding acid. In one embodiment the copolymer has a molecular weight in the range 100,000 to 2,000,000 e.g. from 500,000 to 1,900,000 or from 1,000,000 to 1,800,000. Suitably a copolymer for use in the invention is available commercially under the trade name GANTREZ® such as GANTREZ® S-97 HSU solution (Mw 1,500,000), GANTREZ® S-97 BF (Mw 1,200,000), GANTREZ® S-96 (Mw 700,000) and GANTREZ® S-95 (Mw 150,000), all of which are copolymers of MVE with maleic acid. In one embodiment the copolymer is GANTREZ® S-97 which is a copolymer of MVE with maleic acid having an approximate molecular weight of 1,200,000 or 1,500,000.

GANTREZ® S-97 may be provided in the form of a solid (powder) or as a liquid such as an aqueous solution e.g. GANTREZ® S-97 HSU solution. In one embodiment the copolymer comprises a GANTREZ® polymer with the following structure and below indicated properties:

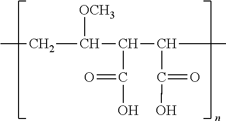

Di-basic acid with pKa₁=3.5, pK a₂=6.5

| | | Gantrez S-97 HSU |
|---|---|---|
| Property | Gantrez S-97 BF | Solution |
| Appearance @ 25° C. | White to off-white, | Slightly hazy |
| | free flowing powder | viscous solution |
| % Solids (Active) | 94 | 15-17 |
| % Moisture | ≤6 | 85-83 |
| Approx. Molecular | 1,200,000 | 1,500,000 |
| Weight | | |

Suitably the rheological properties of the copolymer can be modified by the addition of salts and bases. GANTREZ® copolymers are available commercially from various sources including Ashland Speciality Chemicals, Bound Brook, N.J. 08805, USA and International Specialty Products, Wayne, N.J., USA.

It is a challenge to provide a dentifrice composition that delivers an enhanced fluoridation benefit when the composition comprises a surface protection agent (i.e. a copolymer of use in the invention as hereinabove defined). This is because of surface coverage of sites on the tooth surface by the agent where fluoridation typically takes place. Advantageously in the present invention, the copolymer can be combined with a source of fluoride ions without adversely impacting upon the delivery of fluoride to the dental enamel. It has now been unexpectedly discovered that a low amount of the copolymer provides an improvement with respect to enamel solubility reduction without significantly negatively impacting on fluoride uptake Accordingly, when present, the copolymer is used in an amount from 0.05% to 2% by weight of the composition, such as from 0.1% to 1% or from 0.15% to 0.5% or from 0.2% to 0.4% by weight of the composition. In one embodiment the copolymer is used in an amount of about 0.25% by weight of the composition. It has been surprisingly found in in vitro testing, reported herein, that when a low amount (from 0.2% to 0.3% by weight, exemplified herein by about 0.25% by weight) of copolymer is used, a significant improvement may be observed with respect inhibition of demineralization without adversely affecting fluoride uptake.

These findings have been supported further by the findings of an in-situ erosion study, also reported herein, where a composition according to the invention comprising about 0.25% by weight of a methyl vinyl ether maleic acid copolymer, was seen to outperform all other dentifrice compositions tested, with respect to fluoride uptake, remineralization enhancement and demineralization inhibition. In one embodiment the copolymer is used in amount of about 0.25% by weight of the composition and the composition has a slurry pH of about 6.2.

In one embodiment a composition of the invention does not comprise stannous ions and/or zinc ions. For example in one embodiment a composition of the invention does not comprise from about 0.001% to about 5% of metal ions wherein the metal ions comprise at least 0.001% of stannous ions and optionally from about 0.001% to about 4% of zinc ions. In one embodiment the composition does not comprise a metal compound or salt that becomes more soluble at acidic pH. In one embodiment the composition does not comprise a calcium or zinc compound or salt.

Compositions of the present invention may contain appropriate formulating agents such as dental abrasives, surfactants, thickening agents, humectants, flavouring agents, sweetening agents, opacifying or colouring agents, preservatives and water, selected from those conventionally used in the oral care composition art for such purposes.

Examples of suitable dental abrasives include silica abrasives such as those marketed under the following trade names Zeodent, Sident, Sorbosil or Tixosil by Huber, Degussa, Ineos and Rhodia respectively. The silica abrasive should be present in an amount sufficient to ensure adequate cleaning of teeth by the dentifrice whilst not promoting abrasion of teeth.

The silica abrasive is generally present in an amount up to 15% by weight of the total composition, for example from 2% to 10% by weight, and preferably at least 5% for example from 5% to 7% by weight, especially 6% by weight of the total composition. Reducing the level of silica abrasive has the advantage of not only lowering the abrasivity of the dentifrice but also minimising any interaction of the abrasive with fluoride ions thereby increasing the availability of free fluoride ions.

Suitable surfactants for use in the present invention include amphoteric surfactants for example, long chain alkyl betaines, such as the product marketed under the tradename

‘Empigen BB’ by Albright & Wilson, and preferably long chain alkyl amidoalkyl betaines, such as cocamidopropylbetaine, or low ionic surfactants such as sodium methyl cocoyl taurate, which is marketed under the trade name Adinol CT by Croda, or mixtures thereof. An amphoteric surfactant can be used alone as sole surfactant or can be combined with a low ionic surfactant. In one embodiment the surfactant is not a C10-18 alkyl sulphate surfactant, such as sodium lauryl sulphate, commonly used in oral compositions.

Suitably, the surfactant is present in the range 0.1% to 10%, preferably 0.1% to 5% and more preferably 0.5% to 1.5% by weight of the total composition.

Suitable thickening agents include, for instance, nonionic thickening agents such as, for example, (C1-6)alkylcellulose ethers, for instance methylcellulose; hydroxy(C1-6)alkylcellulose ethers, for instance hydroxyethylcellulose and hydroxypropylcellulose; (C2-6)alkylene oxide modified (C1-6)alkylcellulose ethers, for instance hydroxypropyl methylcellulose; and mixtures thereof. Other thickening agents such as natural and synthetic gums or gum like material such as Irish Moss, xanthan gum, gum tragacanth, sodium carboxymethylcellulose, polyvinyl pyrrolidone, starch and thickening silicas may also be used. Preferably the thickening agent is mixture of a thickening silica and xanthan gum.

Advantageously the thickening agent is present in the range 0.1% to 30%, preferably 1% to 20%, more preferably 5% to 15% by weight of the total composition.

Suitable humectants for use in compositions of the invention include for instance, glycerin, xylitol, sorbitol, propylene glycol or polyethylene glycol, or mixtures thereof;

which humectant may be present in the range from 10% to 80%, preferably 20% to 60%, more preferably 25% to 50% by weight of the total composition.

A preferred opacifying agent is titanium dioxide which may be present in the range 0.05% to 2%, preferably 0.075% to 0.2%, for example 0.1% by weight of the total composition.

This amount enhances the visual appearance of the composition.

Flavouring agents that may be used in a composition of the invention include various flavouring aldehydes, esters, alcohols, and similar materials, as well as menthol, carvone and aethole as well as mixtures thereof. Examples of essential oils include spearmint, peppermint, wintergreen, sassafras, clove, sage, eucalyptus, marjoram, cinnamon, lemon, lime, grapefruit and orange. Suitably the flavouring agent may be used in an amount ranging from 0.01% to 4% such as 0.1% to 3% or 0.5% to 2% by weight of the composition.

Sweetening agents that may be used in a composition of the invention include, for example, sucrose, glucose, saccharin, sucralose, dextrose, levulose, lactose, mannitol, sorbitol, fructose, maltose, xylitol, saccharin salts (e.g. sodium saccharin) acesulfame and mixtures thereof. In one embodiment sodium saccharin is used as the sweetening agent. Suitably the sweetening agent may be used in an amount ranging from 0.005% to 10% such as 0.01% to 3% or 0.1% to 1% by weight of the composition.

Suitably dentifrice compositions of the present invention are aqueous dentifrice compositions. Water may make up the balance of the dentifrice composition. In one embodiment, the composition comprises 5% to 80% such as 10% to 60%, 15% to 40% or 20% to 30% by weight water. This amount of water includes the free water which is added plus that amount which is introduced with other components of the dentifrice composition, such as with sorbitol.

Dentifrice compositions of the present invention are typically formulated in the form of toothpastes or gels.

Additional oral care actives may be included in the compositions of the present invention.

Compositions of the present invention may further comprise a desensitising agent for combating dentine hypersensitivity. Examples of desensitising agents include a tubule blocking agent or a nerve desensitising agent and mixtures thereof, for example as described in WO 02/15809.

Suitable tubule blocking agents include strontium salts such as strontium chloride, strontium acetate or strontium nitrate. Suitably the strontium salt is used in an amount generally from 5% to 15% by weight of the composition.

In one embodiment the tubule blocking agent is an arginine calcium carbonate salt. Suitably the arginine salt is present in an amount ranging from 0.5% to 30% by weight of the composition, such as from 1% to 10% by weight of the composition or from 1% to 10% by weight of the composition such as from 2% to 8% by weight of the composition.

In one embodiment the tubule blocking agent is a bioactive glass. Suitably the bioactive glass consists of 45% by weight silicon dioxide, 24.5% by weight sodium oxide, 6% by weight phosphorus oxide, and 24.5% by weight calcium oxide. One such bioactive glass is available commercially under the trade name, NOVAMIN, also known as 45S5 BIOGLASS. Suitably the bioactive glass is used in an amount generally from 1% to 10% by weight of the composition.

In one embodiment the tubule blocking agent is stannous fluoride. Stannous fluoride, through hydrolysis and oxidation reactions, forms insoluble metal salts that precipitate in dentinal tubules and on the dentine surface to provide effective relief from dentine hypersensitivity. Stannous fluoride may also be used to provide a source of fluoride capable of delivering protection from caries and plaque/gingivitis.

Suitable nerve desensitizing agents include potassium salts such as potassium citrate, potassium chloride, potassium bicarbonate, potassium gluconate and especially potassium nitrate. A desensitising amount of a potassium salt is generally between 2 to 8% by weight of the total composition, for example 5% by weight of potassium nitrate can be used.

Compositions of the present invention may comprise a whitening agent, for example selected from a polyphosphate, e.g. sodium tripolyphosphate (STP) and/or any additional silica abrasive present may have high cleaning properties. STP may be present in an amount from 2% to 15%, for example from 5% to 10% by weight of the total composition.

Compositions of the present invention may comprise an oral malodour agent, for example a zinc salt such as zinc oxide or chloride.

The composition of the present invention is suitable for containing in and dispensing from an aluminium-plastic laminate tube or a plastic pump as conventionally used in the art.

Compositions of the present invention may be prepared by admixing the ingredients in the appropriate relative amounts in any order that is convenient and adjusting the pH to give a desired value.

An exemplary dentifrice composition according to the invention comprises: an alkali metal salt of lactic acid such as sodium lactate in an amount from 0.5% to 5.0%; a source of free fluoride ions such as sodium fluoride in an amount from 0.05% to 0.5%; a copolymer of MVE with maleic anhydride or acid such as GANTREZ° S-97 in an amount from 0.05% to 2%; and wherein the composition has a slurry pH in the range greater than 5.0 to less than 6.5.

The present invention provides a composition as hereinbefore defined for use in protecting teeth against dental erosion. The present invention further provides a composition as hereinbefore defined for use in protecting teeth against dental caries.

The present invention provides a composition as hereinbefore defined for use in the treatment and/or inhibition of dental erosion on a dental surface. The present invention provides a composition as hereinbefore defined for use in the treatment and/or inhibition of caries on a dental surface.

The present invention also provides a method for protecting teeth against dental erosion which comprises applying an effective amount of a composition as hereinbefore defined to an individual in need thereof. The present invention also provides a method for protecting teeth against dental caries which comprises applying an effective amount of a composition as hereinbefore defined to an individual in need thereof.

The present invention provides a method for the treatment and/or inhibition of dental erosion on a dental surface, comprising contacting the dental surface with a composition as hereinbefore defined.

The present invention provides a method for the treatment and/or inhibition of dental caries on a dental surface, comprising contacting the dental surface with a composition as hereinbefore defined. The invention is further illustrated by the following Examples.

### EXAMPLE 1

A dentifrice composition (Formulation I) as described in Table 1 was prepared as follows: To a suitable vessel was added purified water, sorbitol and glycerin. Then sodium hydroxide, sodium lactate solution, sodium saccharin, sodium fluoride, potassium nitrate, Gantrez, titanium dioxide, and 20% of the flavour were added and mixed with high shear until solids were dissolved. Whilst mixing under vacuum the dental silica was added, then mixed until wetted out. The cocamidopropyl betaine solution and the remaining 80% of the flavour were added and mixed. Separately in a pre-mix vessel, the xanthan gum was mixed with approximately 95% of the polyethylene glycol to form a slurry. Under vacuum this slurry was added to the main vessel whilst mixing under high shear. The remainder of the polyethylene glycol was added into the pre-mix vessel and the resulting mixture was flushed into the main vessel. The resulting paste was mixed under vacuum until homogenous then transferred to suitable tubes.

TABLE 1
Formulation 1

| Ingredient Name | % w/w |
|---|---|
| USP Water | 25.7322 |
| Sorbitol (70% w/w) | 30.0000 |
| Silica (thickening + abrasive) | 17.0000 |
| Glycerin | 8.0000 |
| Potassium Nitrate | 5.0000 |
| Sodium Lactate solution (60% w/w) | 4.1466 |
| Polyethylene Glycol | 3.0000 |
| 47% (aq) Cocamidopropyl Betaine Solution | 2.0940 |
| Gantrez S-97 HSU solution (16.5% w/w) | 1.5200 |
| Flavour | 1.2000 |
| Titanium Dioxide | 0.9000 |
| Xanthan Gum | 0.8000 |
| Sodium Saccharin | 0.3000 |
| Sodium Fluoride | 0.2542 |
| Sodium Hydroxide | 0.0530 |
| Total | 100.0000 |

pH of Formulation 1(1:3 slurry in water) = 6.2

### EXAMPLE 2

Enamel Fluoride Uptake (EFU)

This example describes an enamel fluoride uptake study carried out on dentifrice compositions of the invention.

Preparation of Dentifrice Compositions

Formulations 2-4 were prepared having compositional details as provided for in Table 2:

TABLE 2
Compositional Details of Test and Control Dentifrices

| | Formulation 2 | Formulation 3* | Formulation 4* |
|---|---|---|---|
| Ingredient | (Control) | (Test) | (Test) |
| Water | 32.1032 | 39.2348 | 39.3348 |
| Sorbitol (70% w/w) | 30.0000 | 36.0000 | 36.5000 |
| Glycerin | 8.0000 | 2.0000 | 2.0000 |
| PEG 300 (PEG-6) | 3.0000 | 0.4500 | 0.4500 |
| Dental Silica | 18.0000 | 16.0000 | 16.0000 |
| Saccharin Sodium | 0.3000 | 0.3000 | 0.3000 |
| Sodium Fluoride | 0.3152 | 0.3152 | 0.3152 |
| Xanthan gum | 0.8000 | 0.8000 | 0.8000 |
| Carrageenan | — | 0.4000 | 0.4000 |
| Flavour | 1.1000 | 1.0000 | 1.0000 |
| Cocamidopropyl | 1.2000 | 0.8000 | 1.2000 |
| Betaine | | | |
| Titanium Dioxide | 0.1000 | 0.7000 | 0.7000 |
| Potassium Nitrate | 5.0000 | — | — |
| Sodium Hydroxide | 0.0816 | — | — |
| Total | 100.0000 | 98.0000 | 99.0000 |

Formulation 2 was a control composition.
*Formulations 3 and 4 were initial dentifrice compositions that were used in the subsequent preparation of the slurries. Formulations 3 and 4 varied slightly from Formulation 2 to allow for later addition of acid and slurry pH adjustment. Formulations 3 and 4 (see Table 2 above) recite the % w/w amount of each ingredient present in a “final” dentifrice composition, following subsequent addition to the initial dentifrice composition of carboxylic acid and any pH modifying agent required to provide the desired pH.

Preparation of Dentifrice Slurries

Dentifrice slurries were prepared using Formulations 2-4. Slurries were prepared consisting of 1 part paste (Formulation 2, 3 or 4) mixed with 3 parts diluent. The diluent was made of 2 parts acid solution and 1 part water. For the “control”, the acid solution was replaced with water. The total quantity of slurry was 36 g in all cases hence the overall slurry composition consisted of 9 g paste:18 g acid solution:9 g water. This approach was taken to allow the creation of slurries from a common base that would have the correct constitution as if the paste had contained all the ingredients. For example, if Formulation 3 had contained 2% malonic acid, and been mixed with water only, the concentration in the final slurry would have been 0.5% (9 g paste+27 g water, a four-fold dilution). The addition of 18 g of 1% malonic acid solution to 9 g of a base paste containing no malonic acid plus 9 g water also gives a concentration in the final slurry of 0.5% (18 g malonic acid plus a total of 18 g paste and water, a two-fold dilution of the malonic acid solution). The resulting slurries were then centrifuged at 10,000 rpm (˜16,000g) for 10 minutes. The compositional details of the slurries and their respective pH values are provided below in Table 3.

TABLE 3
Composition and pH Details of Dentifrice Slurries

| | | | 1% | 1% | |
|---|---|---|---|---|---|
| | | | Malonic | Citric | |
| | | | Acid | Acid | Slurry |
| Slurry | Dentifrice (9 g) | Water | Soln | Soln | pH |
| 1 | Formulation 2 | 27 g | — | — | 7.2 (unadjusted pH) |
| 2 | Formulation 3 | 9 g | 18 g | — | 7.00 |
| 3 | Formulation 4 | 27 g | — | — | 5.50 |
| 4 | Formulation 3 | 9 g | — | 18 g | 5.50 |
| 5 | Formulation 3 | 9 g | 18 g | — | 5.50 |
| 6 | Formulation 3 | 9 g | 18 g | — | 5.75 |
| 7 | Formulation 3 | 9 g | 18 g | — | 5.25 |

Method

The EFU test procedure was based on Procedure 40 described in the United States Food and Drug Administration (FDA) testing procedures. In the present case, the incipient lesion was formed using 0.1M lactic acid pH 5.0 containing 0.2% w/v polyacrylic acid (Carbopol 907) that was 50% saturated with hydroxyapatite.

Sound, upper, central, bovine incisors were cleaned of all adhering soft tissue. A core of enamel 3mm in diameter was prepared from each tooth using a hollow-core diamond drill bit under running water. Specimens were embedded in the end of a plexiglass rod using methyl methacrylate, and polished with 600 grit wet/dry paper and then with micro-fine Gamma Alumina. Twelve specimens per group were used in the study. Each enamel specimen was etched by immersion into 0.5 ml of 1M perchloric acid (HCl0₄) solution for 15 seconds with continuous agitation.

The fluoride content of this solution was determined by using a fluoride electrode to determine the background fluoride content of the enamel specimens. The specimens were once again ground and polished as described above. An incipient lesion was be formed in each enamel specimen by immersion into a 0.1M lactic acid/0.2% Carbopol 907 solution for 24 hours at 37° C. These specimens were rinsed with water and stored in a humid environment until used.

The pH of certain slurries was adjusted with dropwise addition of 1M hydrochloric acid or 1M sodium hydroxide to achieve the desired pH specified in Table 3. The specimens were immersed into 25 ml of their assigned slurry supernatant with constant stirring (350 rpm) for 30 minutes. Following treatment, the specimens were rinsed with water. One layer of enamel was removed from each specimen by etching as above. The etch solution was analyzed for fluoride (ion-specific electrode) and calcium. The pre-treatment fluoride (indigenous) level of each specimen was then subtracted from the post-treatment value to determine the change in enamel fluoride due to the test treatment.

Statistical Analyses

Statistical analyses of the individual means were performed with a one-way analysis of variance model. Significance of differences was analyzed by the Student Newman-Keuls test.

Results

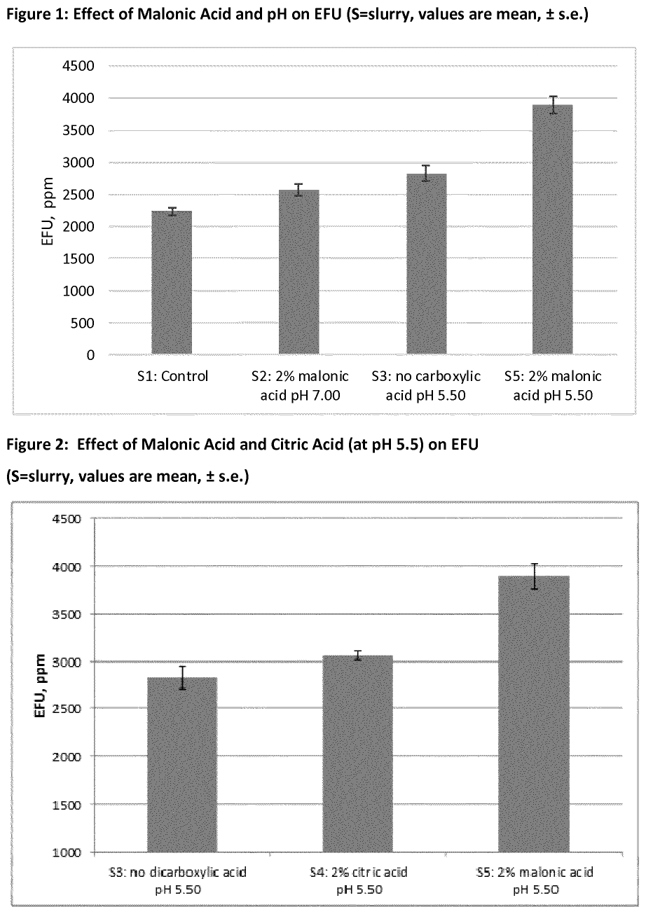

The results of the study are presented in Table 4 (mean EFU±standard error of the mean) and FIGS. 1-3 below.

TABLE 4
Results of EFU Study

| Slurry | Treatment | EFU | s.e. |
|---|---|---|---|
| 1 | Formulation 2 (Control) | 2236 | 58 |
| 2 | Formulation 3 pH 7.00 2% malonic acid | 2575 | 99 |
| | (Control) | | |
| 3 | Formulation 4 pH 5.50 no carboxylic acid | 2826 | 118 |
| | (Control) | | |
| 4 | Formulation 3 pH 5.50 2% citric acid | 3062 | 51 |
| | (Control) | | |
| 5 | Formulation 3 pH 5.50 2% malonic acid | 3895 | 133 |
| | (Test slurry) | | |
| 6 | Formulation 3 pH 5.75 2% malonic acid | 3719 | 129 |
| | (Test slurry) | | |
| 7 | Formulation 3 pH 5.25 2% malonic acid | 3919 | 127 |
| | (Test slurry) | | |

In FIG. 1, at the 5% significance level, all treatments were statistically significantly different to each other. A modest benefit was observed for including the malonic acid at neutral pH, and a slightly greater benefit was observed by reducing the pH to pH 5.5 without adding carboxylic acid by dropwise addition of 1M HCl. By combining the two—pH 5.5 plus carboxylic acid—a substantially greater benefit was observed than either alone demonstrating an unexpected synergy of reducing pH and adding particular carboxylic acid.

In FIG. 2, the effect of malonic acid at 2% at pH 5.5 was much greater than the effect of citric acid at 2% pH 5.5, demonstrating an unexpected dependence on the nature of the acid used.

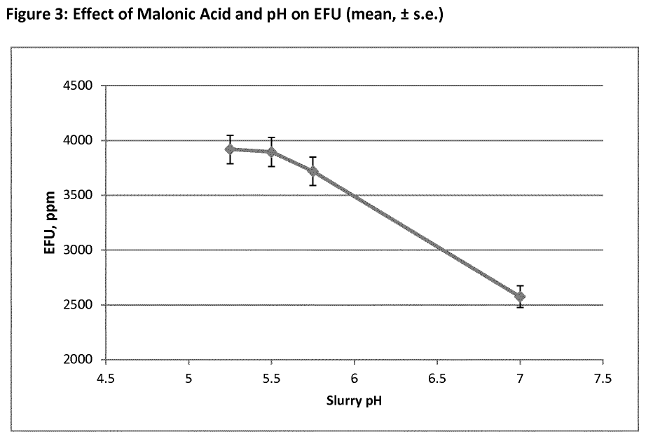

In FIG. 3, as the pH was decreased the EFU rose until pH 5.5 was reached. There was no further increase in EFU by reducing the pH 5.5 to pH 5.25.

Conclusion

Synergistic benefit on EFU was observed by reducing the pH to pH 5.5 and adding the carboxylic acid, malonic acid, at 2%. The maximum benefit to EFU in a 2% carboxylic acid was observed at pH 5.5 for malonic acid: below this value EFU did not increase. The boost to EFU from inclusion of malonic acid in these conditions was much greater than the boost from including citric acid.

### EXAMPLE 3

Enamel Fluoride Uptake (EFU)

This example describes an enamel fluoride uptake study carried out on dentifrice compositions of the invention.

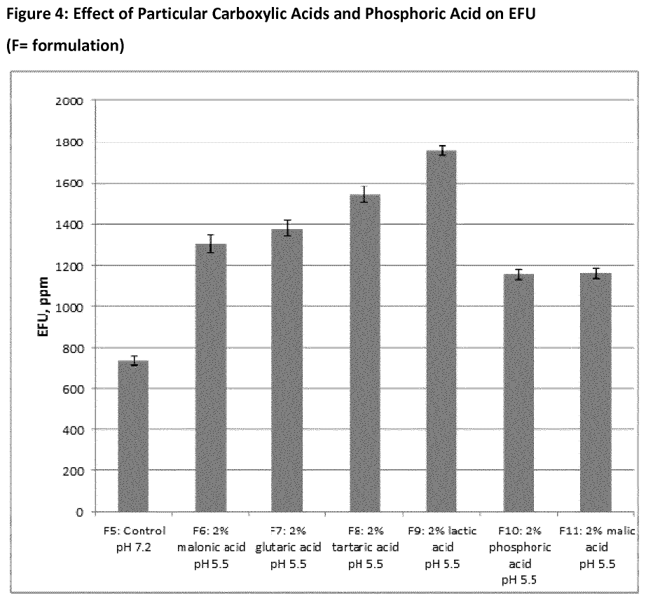

Dentifrice compositions (Formulations 5-11) were prepared (see Table 5 below) and EFU determined as described in Example 2 above. The results are shown in Table 6, and FIG. 4.

TABLE 5
Compositional Details of Test and Control Dentifrices

| | Formulation | | | | | | |
|---|---|---|---|---|---|---|---|
| | 5 | | | | | 10 | 11 |
| | (Control) | 6 | 7 | 8 | 9 | (Control) | (Control) |
| Ingredients: | % w/w | % w/w | % w/w | % w/w | % w/w | % w/w | % w/w |
| Water | 32.1034 | 37.465 | 37.685 | 39.335 | 39.335 | 39.335 | 37.795 |
| Sorbitol (70% w/w) | 30.0000 | 36.000 | 36.000 | 35.500 | 35.500 | 35.500 | 36.000 |
| Glycerin | 8.0000 | 2.000 | 2.000 | 2.000 | 2.000 | 2.000 | 2.000 |
| PEG 300 (PEG-6) | 3.0000 | 0.450 | 0.450 | 0.450 | 0.450 | 0.450 | 0.450 |
| Dental Silica | 18.0000 | 16.000 | 16.000 | 16.000 | 16.000 | 16.000 | 16.000 |
| Saccharin, sodium | 0.3000 | 0.300 | 0.300 | 0.300 | 0.300 | 0.300 | 0.300 |
| Sodium fluoride | 0.3150 | 0.315 | 0.315 | 0.315 | 0.315 | 0.315 | 0.315 |
| Xanthan gum | 0.8000 | 0.800 | 0.800 | 0.800 | 0.800 | 0.800 | 0.800 |
| Carrageenan | — | 0.400 | 0.400 | 0.400 | 0.400 | 0.400 | 0.400 |
| Flavour | 1.1000 | 1.000 | 1.000 | 1.000 | 1.000 | 1.000 | 1.000 |
| Cocamidopropyl | 1.2000 | 1.200 | 1.200 | 1.200 | 1.200 | 1.200 | 1.200 |
| Betaine | | | | | | | |
| Titanium Dioxide | 0.1000 | 0.700 | 0.700 | 0.700 | 0.700 | 0.700 | 0.700 |
| Potassium Nitrate | 5.0000 | — | — | — | — | — | — |
| Malonic acid, solid | — | 2.000 | — | — | — | — | — |
| Glutaric acid | — | — | 2.000 | — | — | — | — |
| Malic acid | — | — | — | — | — | — | 2.000 |
| Tartaric Acid | — | — | — | 2.000 | — | — | — |
| Lactic Acid | — | — | — | — | 2.000 | — | — |
| Potassium dihydrogen | — | — | — | — | — | 2.000 | — |
| phosphate | | | | | | | |
| Sodium hydroxide, | 0.0816 | 1.370 | 1.150 | — | — | — | 1.040 |
| solid | | | | | | | |
| Total | 100.0000 | 100.000 | 100.000 | 100.000 | 100.000 | 100.000 | 100.000 |

Results

TABLE 6
Results of EFU Study

| # | T reatment | EFU | s.e. |
|---|---|---|---|
| 1 | Formulation 5 (Control) (pH 7.2) | 733 | 23.6 |
| 2 | Formulation 6 2% malonic acid pH 5.5 | 1305 | 42 |
| 3 | Formulation 7 2% glutaric acid pH 5.5 | 1379 | 40.2 |
| 4 | Formulation 8 2% tartaric acid pH 5.5 | 1544 | 41.5 |
| 5 | Formulation 9 2% lactic acid pH 5.5 | 1754 | 22.1 |
| 6 | Formulation 10 2% phosphoric acid* pH | 1154 | 26.4 |
| | 5.5 (Control) | | |
| 7 | Formulation 11 2% malic acid pH 5.5 | 1157 | 27 |
| | (Control) | | |

*added as potassium dihydrogen phosphate

At the 5% significance level, all treatments with added acid at pH 5.5 had EFU values statistically significantly greater than the acid-free toothpaste at pH 7.2. The 2% lactic acid product was superior to all other treatments, followed by the 2% tartaric acid product.

The EFU values for the phosphoric acid example and the malic acid example were significantly lower than those observed with the carboxylic acids of use in the invention.

Conclusion

When added to a toothpaste at 2% w/w at pH 5.5, different acids exerted substantially different effect on EFU. Lactic acid was the most effective of those tested. The results according to this study demonstrate that a significant effect with respect to fluoride uptake is not achieved merely by formulating a dentifrice composition at an acidic pH (5.5) nor is it achieved merely by using any carboxylic acid. The results observed with phosphoric acid and malic acid were significantly less impressive compared to those observed with carboxylic acids of use in the invention.

### EXAMPLE 4

Enamel Fluoride Uptake (EFU)

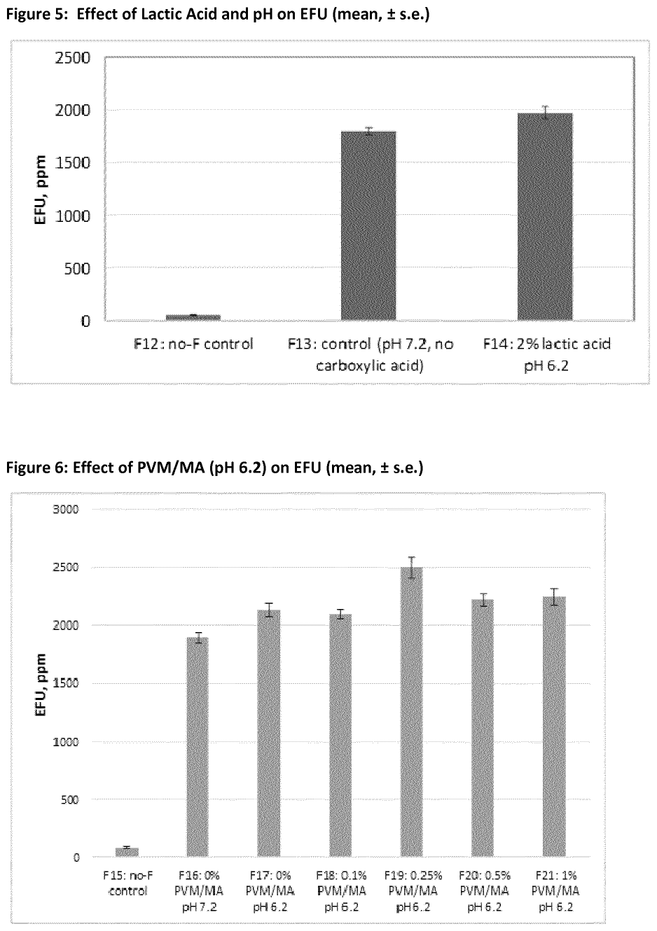

Dentifrice compositions Formulations 12-14 described below (See Table 7) were prepared and EFU determined as described in Example 2 above. The results are shown in Table 8, and FIG. 5.

TABLE 7
Formulations 12-14

| | Formulation | | |
|---|---|---|---|
| | 12 | 13 | |
| | (Control) | (Control) | 14 |
| Ingredient | % w/w | % w/w | % w/w |
| Water | 25.5194 | 30.4518 | 25.2652 |
| Sorbitol (70% w/w) | 30.0000 | 30.0000 | 30.0000 |
| Dental Silica | 17.0000 | 18.0000 | 17.0000 |
| Glycerin | 8.0000 | 8.0000 | 8.0000 |
| Potassium Nitrate | 5.0000 | 5.0000 | 5.0000 |
| PEG 300 (PEG-6) | 3.0000 | 3.0000 | 3.0000 |
| Sodium Lactate | 4.1466 | — | 4.1466 |
| solution (60% w/w) | | | |
| PVM/MA* Copolymer | 1.5200 | — | 1.5200 |
| 16.5% Solution | | | |
| Saccharin Sodium | 0.3000 | 0.3000 | 0.3000 |
| Sodium Fluoride | — | 0.2542 | 0.2542 |
| Xanthan gum | 0.8000 | 0.8000 | 0.8000 |
| Flavour | 1.2000 | 1.2000 | 1.2000 |
| 47% w/w | 2.0940 | 2.0940 | 2.0940 |
| Cocamidopropyl | | | |
| Betaine solution | | | |
| Titanium Dioxide | 0.9000 | 0.1000 | 0.9000 |
| 10.2% Sodium | 0.5200 | 0.8000 | 0.5200 |
| Hydroxide solution | | | |

*PVM/MA = polyvinyl methyl ether/maleic acid

Results

TABLE 8
Results of EFU Study

| Formulation | EFU | s.e. |
|---|---|---|
| Formulation 12 (Control - no | 52 | 6 |
| fluoride) | | |
| Formulation 13 (Control - no | 1800 | 38 |
| carboxylic acid salt or copolymer) | | |
| Formulation 14 | 1978 | 59 |

Conclusion

Formulation 14 was superior to the fluoride control formulation. Both fluoride-containing formulations were superior to the fluoride-free control formulation.

### EXAMPLE 5

### EFU Study

Dentifrice compositions Formulations 15-21 described below (see Table 9) were prepared and EFU determined as described in Example 2 above. The results are shown in Table 10, and FIG. 6.

TABLE 9
Formulations 15-21

| | Formulation | | | | | | |
|---|---|---|---|---|---|---|---|
| | 15 | 16 | 17 | 18 | 19 | 20 | 21 |
| Ingredients | % w/w | % w/w | % w/w | % w/w | % w/w | % w/w | % w/w |
| Water | 31.5971 | 31.4458 | 31.2522 | 31.29 | 30.03 | 27.92 | 23.69 |
| Sorbitol (70% w/w) | 30.0000 | 30.0000 | 30.0000 | 30.00 | 30.00 | 30.00 | 30.00 |
| Dental Silica | 18.0000 | 18.0000 | 18.0000 | 18.00 | 18.00 | 18.00 | 18.00 |
| Glycerin | 8.0000 | 8.0000 | 8.0000 | 8.00 | 8.00 | 8.00 | 8.00 |
| Potassium Nitrate | 5.0000 | 5.0000 | 5.0000 | 5.00 | 5.00 | 5.00 | 5.00 |
| PEG 400 (PEG-8) | 3.0000 | 3.0000 | 3.0000 | 3.00 | 3.00 | 3.00 | 3.00 |
| Cocamidopropyl | 1.2000 | 1.2000 | 1.2000 | 1.20 | 1.20 | 1.20 | 1.20 |
| Betaine | | | | | | | |
| Flavour | 1.2000 | 1.1000 | 1.2000 | 1.20 | 1.20 | 1.20 | 1.20 |
| Xanthan gum | 0.8000 | 0.8000 | 0.8000 | 0.80 | 0.80 | 0.80 | 0.80 |
| Saccharin, sodium | 0.3000 | 0.3000 | 0.3000 | 0.30 | 0.30 | 0.30 | 0.30 |
| Sodium fluoride | — | 0.2542 | 0.2542 | 0.25 | 0.25 | 0.25 | 0.25 |
| Titanium Dioxide | 0.1000 | 0.1000 | 0.1000 | 0.10 | 0.10 | 0.10 | 0.10 |
| PVM/MA* Copolymer | — | — | — | 0.61 | 1.52 | 3.03 | 6.06 |
| 16.5% solution | | | | | | | |
| (Gantrez S-97) | | | | | | | |
| 10.2% NaOH solution | 0.8039 | 0.8000 | — | 0.25 | 0.60 | 1.20 | 2.40 |
| Total | 100.0000 | 100.0000 | 100.0000 | 100.00 | 100.00 | 100.00 | 100.00 |

*PVM/MA = polyvinyl methyl ether/maleic acid

Results

TABLE 10
EFU values of dentifrice compositions
comprising PVM/MA Copolymer

| Formulation | EFU | s.e. |
|---|---|---|
| Formulation 15 | 86 | 7 |
| Formulation 16 (pH 7.2) | 1896 | 50 |
| Formulation 17 | 2136 | 62 |
| (adjusted to pH 6.2) | | |
| Formulation 18 | 2096 | 45 |
| (adjusted to pH 6.2) | | |
| Formulation 19 | 2498 | 87 |
| (adjusted to pH 6.2) | | |
| Formulation 20 | 2219 | 55 |
| (adjusted to pH 6.2) | | |
| Formulation 21 | 2249 | 73 |
| (adjusted to pH 6.2) | | |

At 5% significance level, all fluoride-containing formulations were statistically significantly greater than the fluoride-free placebo. The formulation containing 0.25% PVM/MA copolymer (Formulation 19) was statistically significantly superior to all other formulations tested. There were no significant differences between the other formulations.

Conclusion

All fluoride-containing formulations were superior to the fluoride-free placebo. However there was evidence to suggest that use of 0.25% polymer was surprisingly favourable to EFU.

### EXAMPLE 6

### Enamel Solubility Reduction Study

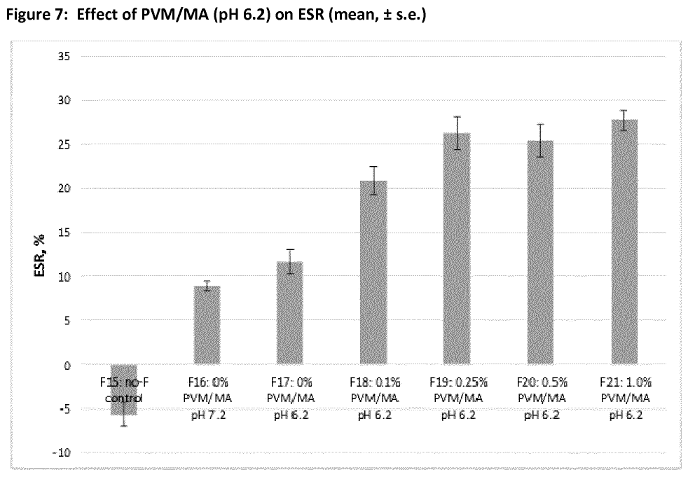

Dentifrice compositions Formulations 15-21 described above in Table 10 were prepared and ESR determined as described below. The results are shown in Table 11, and FIG. 7.

Tooth Preparation

Three sound human molars were placed in wax so that only the enamel surfaces were exposed, then cleaned and polished. Twelve sets of three teeth each were prepared for the study.

Lactate Buffer Preparation

A 0.1 M lactic acid solution buffered to pH 4.5 was prepared.

Deprotection

Teeth surfaces were etched in 0.1 M lactate buffer solution for two one-hour periods at room temperature, then rinsed well with water.

Pre-Treatment Etch

The test was performed using preheated (37° C.) tooth sets and lactate buffer in an incubator. The acid-pre-treated teeth sets were mounted on the ends of acrylic rods with molten wax. A small hole was drilled in each container lid to accommodate the plastic rod to which the tooth sets were mounted. A 40 ml portion of 0.1 M lactic acid buffer was placed in each container. The rod of the first tooth set will be pushed through the hole in the lid, placed in the first container and adjusted so that all enamel surfaces were immersed into the lactic acid solution. After 15 minutes of stirred exposure to the buffered lactate solution, the tooth sets were removed from the container and rinsed in water. The lactate buffer solutions were retained and analysed for phosphorus. The tooth sets were then placed back in the 37° C. water bath in preparation for the treatment step.

Treatment

All tooth sets were treated at the same time (one for each product). The treatment procedure was similar to the etching procedure with the exception of the dentifrice slurry in place of the acid. A 30 ml portion of preheated dentifrice slurry was added to each container, then the teeth were immersed in the dentifrice slurry and stirred for 5 minutes. The other tooth sets were treated in the same manner with the other dentifrice slurries. At the end of treatment, the tooth sets were removed and rinsed well with water

Post-Treatment

A second lactic acid exposure was performed by the same method as the pre-treatment etch on the dentifrice-treated samples and the treatment solutions analysed for phosphorus. The pre- and post-treatment solutions were analyzed for phosphorus using a Klett-Summerson Photelectric Colorimeter.

The tooth sets were the etched again and the procedure repeated additional times so that each tooth set was treated with each dentifrice. The treatments were allocated in a Latin Square design to ensure treatment sequences varied.

Calculation of E.S.R.

The percent of enamel solubility reduction was calculated as the difference between the amount of phosphorus in the pre- and post-acidic solutions, divided by the amount of phosphorus in the pre-solution, multiplied by 100.

Results

TABLE 11
Results of ESR Study

| # | Sample ID | ESR | s.e. |
|---|---|---|---|
| 1 | Formulation 15 | −5.68 | 1.41 |
| 2 | Formulation 16 | 8.94 | 0.53 |
| 3 | Formulation 17 | 11.67 | 1.37 |
| | (adjusted to pH 6.2) | | |
| 4 | Formulation 18 | 20.86 | 1.59 |
| | (adjusted to pH 6.2). | | |
| 5 | Formulation 19 | 26.23 | 1.77 |
| | (adjusted to pH 6.2) | | |
| 6 | Formulation 20 | 25.43 | 1.86 |
| | (adjusted to pH 6.2) | | |
| 7 | Formulation 21 | 27.68 | 1.15 |
| | (adjusted to pH 6.2) | | |

Results

All fluoride-containing dentifrices gave ESR values statistically superior to the fluoride-free placebo. A clear dose-response to PVM/MA copolymer content was observed between 0% and 0.25%. An approximately 15% increase in ESR was observed due to the presence of 0.25% PVM/MA copolymer. Above 0.25%, no further increase in ESR was observed up to at least 1% PVM/MA copolymer.

Conclusion

Addition of PVM/MA copolymer up to 0.25% caused a significant increase in enamel solubility reduction. No further increase was noted on adding higher levels of the copolymer.

### EXAMPLE 7

Introduction

In order to evaluate the effectiveness of the test formulation a clinical in situ study was performed to compare the effectiveness of the test formulation against a fluoride-free placebo control and a comparator toothpaste also indicated for enamel erosion. The study design employed here has previously been extensively used to investigate the performance of formulations in remineralising acid-softened enamel [Creeth, 2018; Zero, 2006; Barlow, 2009; Creeth, 2015].

The protocol for the study was posted on the ClinicalTrials.gov website on the 28 Sep. 2017 (Clinicaltrials.gov (Identifier: NCT03296072)).

Formulations

The test formulation, Formulation 1, is described in Example 1. The fluoride-free placebo was an identical formula to the test, but with the fluoride replaced with water, and the comparator toothpaste was Crest ProHealth Sensitivity and Enamel Shield.

Study Details

This study was a single centre, controlled, single-blind (to the dental examiner and specimen analysts), randomised, three-treatment, three-period, cross-over in situ design to test the remineralising performance of dentifrices. Treatment was provided once and assessed 2 and 4 hours after application. A washout phase of 2 days (using a fluoride-free dentifrice) was implemented prior to each treatment visit.

In this study, subjects were fitted with an intra-oral device that was capable of holding 8 enamel specimens palatally in the mouth. The enamel specimens were cut from bovine permanent incisors and serially polished to a mirror finish. The specimens were demineralised in vitro by contacting with grapefruit juice for 25 minutes. Specimens were then mounted in the intra-oral appliances and worn by the subjects for the duration of the test period. The toothpaste treatments were brushed onto the buccal surfaces of the teeth for 25 s, then the resulting slurry was swilled around the mouth for 95 s, expectorated and rinsed with water. Four enamel specimens were removed from the appliance 2 hours after treatment, with the remaining 4 specimens removed 4 hours after treatment. The enamel was then immersed in grapefruit juice in vitro a second time. The amount of remineralisation that had occurred was determined through measuring the microhardness of the enamel surface using a Knoop micro indenter. Indents were performed on the sound enamel prior to contact with grapefruit juice, prior to insertion in the mouth, after the 2 or 4 hours remineralisation period and after the second grapefruit juice challenge. The length of the indents was used to calculate the percentage surface microhardness recovery (% SMHR) and the percentage relative erosion resistance (% RER):

% SMHR=[(E1-R)/(E1-B)]*100 [from Gelhard, 1979]

% RER=[(E1-E2)/(E1-B)]*100[from Corpron, 1986]

where B=indentation length (μm) of sound enamel at baseline; E1=indentation length (μm) after first grapefruit juice challenge; R=indentation length (μm) after in situ remineralization and E2=Indentation length (μm) after the second grapefruit juice challenge.

The amount of fluoride incorporated into the remineralised lesion (Enamel fluoride uptake (EFU)) was also chemically determined (using the method of Sakab [Sakkab 1984]) after the enamel specimens had been removed from the mouth, but prior to the second grapefruit juice challenge.

Results

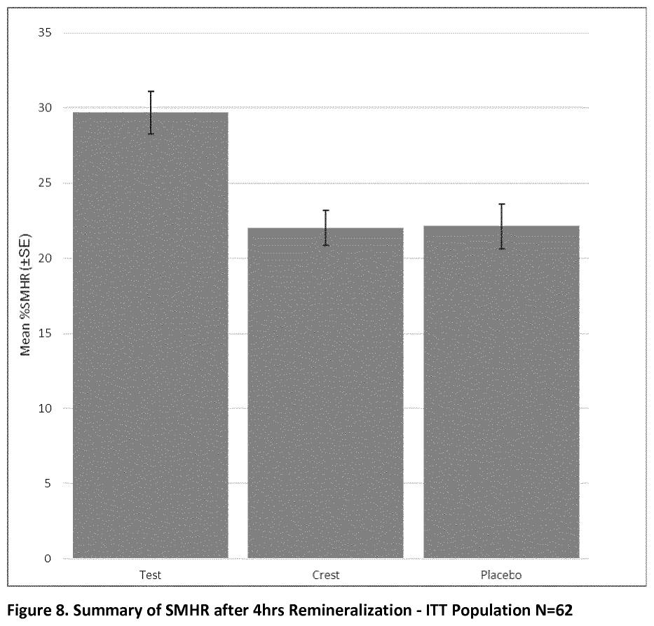

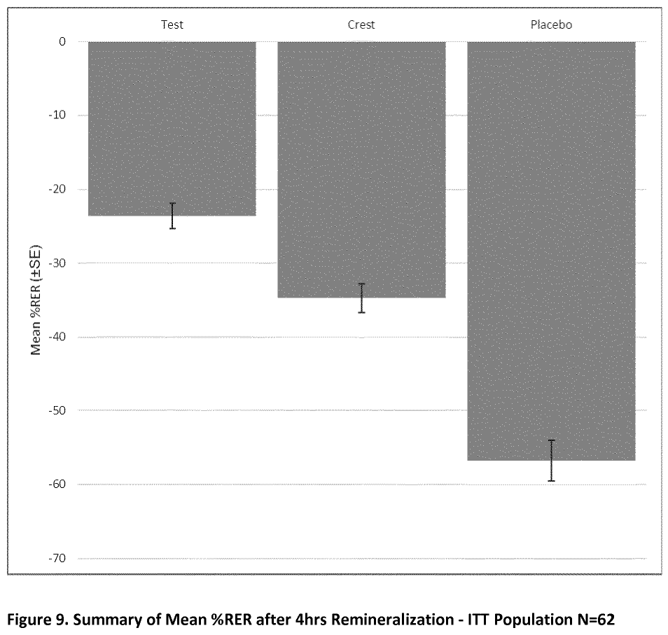

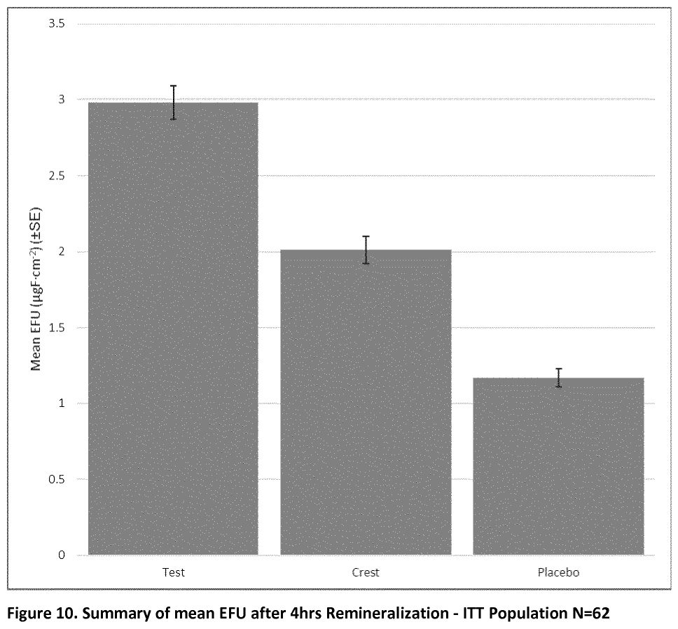

The results are shown in FIGS. 8-10. The test toothpaste showed statistically significantly greater remineralisation (as demonstrated by the % SMHR) than either the placebo control or the comparator toothpaste. The test toothpaste also showed statistically superior prevention of demineralisation (as shown by % RER) than either the placebo or the comparator toothpaste. In addition, the enamel treated with the test toothpaste had incorporated into the remineralising lesion (EFU) was statistically superior to the enamel treated with either the fluoride free placebo or the comparator toothpaste.

Conclusion

The results show that the test toothpaste was more effective at remineralising acid-softened enamel and at preventing further demineralisation than either a fluoride-free control or a comparator product indicated for erosion.

### REFERENCES

Barlow A P, Sufi F, Mason S C. Evaluation of different fluoridated dentifrice formulations using an in-situ erosion remineralization model. The Journal of Clinical Dentistry. 2009;20(6):192-8.
Corpron R E, Clark J W, Tsai A, More F G, Merrill D F, Kowalski C J, Tice T R, Rowe C E. Intraoral effects of a fluoride-releasing device on acid-softened enamel. The Journal of the American Dental Association. 1986 Sep. 1; 113(3):383-8.
Creeth J E, Kelly S A, Martinez-Mier E A, Hara A T, Bosma M L, Butler A, Lynch R J, Zero D T. Dose-response effect of fluoride dentifrice on remineralisation and further demineralisation of erosive lesions: A randomised in situ clinical study. Journal of Dentistry. 2015 Jul. 1; 43(7):823-31.
Creeth J E, Parkinson C R, Burnett G R, Sanyal S, Lippert F, Zero D T, Hara A T. Effects of a sodium fluoride-and phytate-containing dentifrice on remineralisation of enamel erosive lesions—an in situ randomised clinical study. Clinical oral investigations. 2018 Feb. 8:1-0. Gelhard T B, Ten Cate J M, Arends J. Rehardening of artificial enamel lesions in vivo. Caries Research. 1979; 13(2):80-3.
Sakkab N Y, Cilley W A, Haberman J P. Fluoride in deciduous teeth from an anti-caries clinical study. Journal of Dental Research. 1984 October; 63(10):1201-5.
Zero D T, Hara A T, Kelly S A, Gonzalez-Cabezas C, Eckert G J, Barlow A P, Mason S C. Evaluation of a desensitizing test dentifrice using an in-situ erosion remineralization model. The Journal of Clinical Dentistry. 2006; 17(4):112-6.

### EXAMPLE 8

### White Light Inteferometry Analysis (Enamel Protection)

Introduction

The aim of this study was to monitor and quantify the effect, in vitro, of treating human enamel with dentifrice formulations on subsequent erosion by a dietary acid. The technique of White Light Interferometry can provide rapid visualisation of surface topography. Determination of roughness parameters can be carried out in non-contact mode, and height resolution on the nanometer scale is obtainable.

Test Products

| T1 | Composition of Example 1, Formulation 1 |
|---|---|
| C1 | Competitor toothpaste comprising stannous fluoride and no Gantrez |
| | polymer (Comparator formulation) |
| C2 | Competitor toothpaste comprising sodium fluoride and no Gantrez |
| | polymer (Comparator formulation) |
| C3 | Placebo toothpaste for Formulation 1 which is fluoride-free and |
| | Gantrez -free |

C1—Crest Prohealth Smooth Formula Toothpaste (Ingredients: Stannous Fluoride 0.454% (0.14% W/V Fluoride Ion), Water, Sorbitol, Hydrated Silica, Sodium Lauryl Sulfate, Carrageenan, Sodium Gluconate, Flavor, Xanthan Gum, Zinc Citrate, Stannous Chloride, Sodium Hydroxide, Sodium Saccharin, Sucralose, Titanium Dioxide, Blue 1)
C2—Colgate Enamel Health Toothpaste (Ingredients: Potassium nitrate 5%, Sodium fluoride 0.24% (0.15% w/v fluoride ion) water, sorbitol, hydrated silica, glycerin, PEG-12, tetrasodium pyrophosphate, sodium lauryl sulfate, flavor, microcrystalline cellulose, zinc phosphate, cellulose gum, cocamidopropyl betaine, benzyl alcohol, sodium saccharin, xanthan gum, mica, titanium dioxide, FD&C blue no. 1).

Methodology

Twenty Human Enamel specimens were polished flat and a region of their surface taped off using acid resistant tape. The specimens were then divided into four treatment groups (n=5 for each group) and immersed into one of dentifrice slurries (1:3 wt % in deionised water) with manual brushing for 2 minutes. Samples were then washing for 1 minute with deionised water. After dentifrice treatment, specimens were suspended in 1% citric acid, pH 3.8 for 5 minutes, without agitation. Specimens were washed with deionised water and air dried then analysed using white light interferometry.

The surface topography of the specimens was investigated using an ADE PhaseShift

MicroXAM White Light Interferometer. Data was acquired from multiple areas (of size 687 μm×511 um and 215 μm×160 μm) for each specimen. After removal of the tape mask, additional measurements were made to assess bulk tissue loss. Statistical analysis was carried out using a two tailed, unequal variance Student T-Test to >95% confidence level.

Results

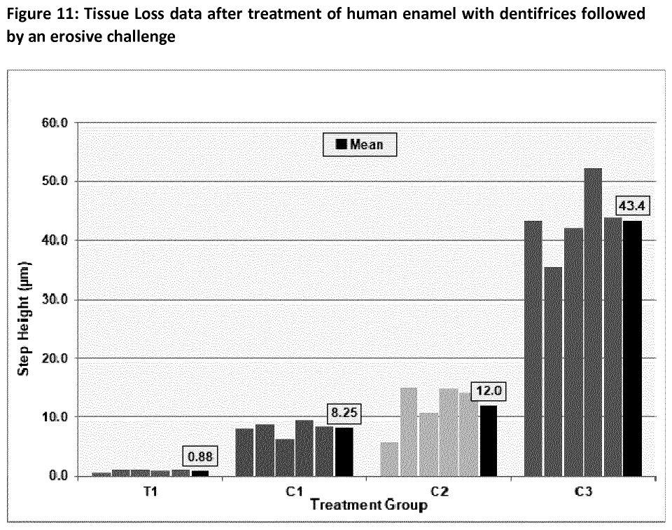

The results are shown in FIG. 11.

The material loss for the treatment groups followed the trend:

[Largest Step]C3>C2>C1>T1 [Smallest Step]. The step height differences between all treatment groups are statistically significant at a 95% confidence level.

The surface roughness for the treatment groups followed the trend:

[Largest Sa]C3>C2>C1>T1[Smallest Step].pair. The Sa differences between all treatment groups are statistically significant at a 95% confidence level except in the case of C2 and C1.

Conclusion

The above data indicate that pre-treatment with T1 offer the greatest protection against an erosive challenge, followed by pre-treatment with C1, followed by pre-treatment with C2, with the lowest protection offered by pretreatment with C3.

### EXAMPLE 9

### Dynamic Secondary Ion Mass Spectrometry (Fluoride Uptake)

Introduction

Dynamic Secondary Ion Mass Spectrometry (DSIMS) can be used to semi-quantitatively determine elemental depth profiles of materials, with nanometre scale resolution. This technique has been used to determine the extent of fluoride and calcium uptake into human enamel surfaces after treatment of erosive lesions with dentifrices and mouthrinses. The aim of this study was to determine the extent of fluoride uptake into human enamel artificial erosive lesions after treatment with the four dentifrices investigated in the white light interferometry study detailed above.

Twenty Human Enamel specimens were polished and suspended in 1% citric acid, pH 3.8 for 5 minutes, without agitation to create artificial erosive lesions. After washing with deionised water, specimens were divided into 4 treatment groups (n=5) and immersed into dentifrice slurries (1:3 wt %) for 2 minutes before washing for 1 minute with deionised water. After treatment, specimens were air dried and analysed using fluoride DSIMS.

DSIMS imaging analysis was carried out with a Cameca ims 6f instrument using a 15 keV O₂⁺ primary ion beam (˜50 pA) and an electron gun for charge compensation. Images were acquired from areas measuring 100 um×100 um. Negative secondary ion detection was used with a nominal extraction field of −5.0 keV. Fluorine/Oxygen integral values were determined for a depth range of 50 μm, i.e. a measure of the relative uptake of fluoride into the upper 50 μm of the tooth enamel surface. A graphical comparison of the results of fluoride uptake across the four treatment groups is shown in FIG. 3.

Test Products (same as for Example 8)

| T1 | Composition of Example 1, Formulation 1 |
|---|---|
| C1 | Competitor toothpaste comprising stannous fluoride and no Gantrez |
| | polymer (Comparator formulation) |
| C2 | Competitor toothpaste comprising sodium fluoride and no Gantrez |
| | polymer (Comparator formulation) |
| C3 | Placebo toothpaste for Formulation 1 which is fluoride-free and |
| | Gantrez -free |

Methodology

Twenty Human Enamel specimens were polished and suspended in 1% citric acid, pH 3.8 for 5 minutes, without agitation to create artificial erosive lesions. After washing with deionised water, specimens were divided into 4 treatment groups (n=5) and immersed into dentifrice slurries (1:3 wt %) for 2 minutes before washing for 1 minute with deionised water. After treatment, specimens were air dried and analysed using fluoride DSIMS.

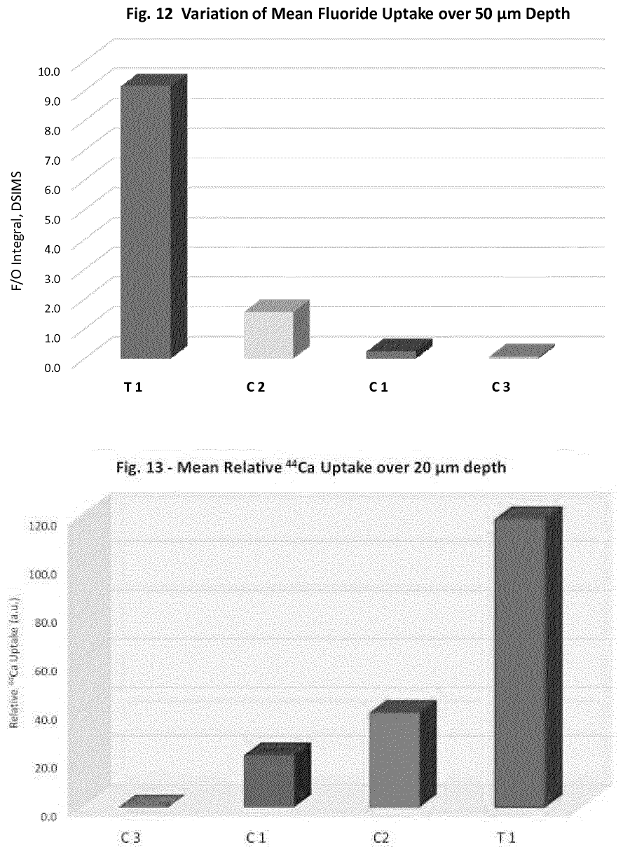

DSIMS imaging analysis was carried out with a Cameca ims 6f instrument using a 15 keV O₂⁺ primary ion beam (˜50 pA) and an electron gun for charge compensation. Images were acquired from areas measuring 100 μm×100 μm. Negative secondary ion detection was used with a nominal extraction field of −5.0 keV. Fluorine/Oxygen integral values were determined for a depth range of 50 μm, i.e. a measure of the relative uptake of fluoride into the upper 50 μm of the tooth enamel surface. A graphical comparison of the results of fluoride uptake across the four treatment groups is shown in FIG. 12.

Results

The results of fluoride DSIMS analysis and retrospective line scan analysis showed that fluoride uptake is greatest for specimens treated with the T1 dentifrice, followed by the C2, followed by the C1 dentifrice. Treatment with the C3 dentifrice lead to very little fluoride uptake. In order to assess any statistically significant differences in fluoride uptake between the treatment groups, a Student “T” Test was carried out. All differences between the treatment groups were found to be statistically significant.

### EXAMPLE 10

### Dynamic Secondary Ion Mass Spectrometry (Calcium Uptake)

Introduction

The aim of this study was to determine the extent of calcium uptake into human enamel artificial erosive lesions after treatment with three dentifrices.

Test Products (Same as for Example 8)

| T1 | Composition of Example 1, Formulation 1 |
|---|---|
| C1 | Competitor toothpaste comprising stannous fluoride and no Gantrez |
| | polymer (Comparator formulation) |
| C2 | Competitor toothpaste comprising sodium fluoride and no Gantrez |
| | polymer (Comparator formulation) |
| C3 | Placebo toothpaste for Formulation 1 which is fluoride-free and |
| | Gantrez -free |

Methodology

Twenty Human Enamel specimens were polished and suspended in 1% citric acid, pH 3.8 for 5 minutes, without agitation. After washing with deionised water, specimens were then divided into 4 treatment groups (n=5) and immersed into dentifrice slurries (1:3 wt %) for 2 minutes before washing for 1 minute with deionised water. Enamel specimens from two of the four treatment groups were incubated in the slurry made from the dentifrice T1. The enamel was subsequently placed into an artificial saliva solution for 24 hrs. This solution contained calcium significantly enriched with ⁴⁴calcium (as calcium chloride) for three of the treatments. For enamel in the second dentifrice C3 (placebo for T1) a standard artificial saliva solution was used as a control (identical to the artificial saliva used for the other treatments but containing ⁴⁰calcium as calcium chloride).

Specimens were then washing for 1 minute with deionised water, air dried and analysed using for ⁴⁴calcium DSIMS. DSIMS imaging analysis was carried out with a Cameca ims 4F instrument utilising a 15 keV O2+ primary ion beam (˜100 pA). Images for the species ⁴⁰Ca, ⁴²Ca, ⁴⁴Ca and ⁴⁰Ca¹⁹F were acquired from a minimum of two areas per sample measuring, typically, 100μm×100 μm. Positive secondary ion detection was used with an extraction field at the sample surface of +4.5 keV and a normal incidence electron gun for charge compensation. Linescan data was subsequently obtained from each image using the Cameca ims 4f data processing software. A graphical representation of the results is shown in FIG. 13.

Results

DSIMS imaging of the enamel and retrospective line scan analysis showed that ⁴⁴calcium uptake was negligible in the specimens treated with C3 but subsequently incubated in the artificial saliva solution composed of calcium of normal isotopic composition. For the specimens incubated in the ⁴⁴calcium-enriched artificial saliva, the extent of ⁴⁴calcium incorporation was greatest for specimens pre-treated with the T1 dentifrice, followed by those treated with the C2 dentifrice, followed by the C1 dentifrice (FIG. 13). Although ⁴⁴calcium uptake occurs to a depth of greater than 20 μm for the first three treatments, the greatest inter-group differences in mean ⁴⁴calcium uptake occur in the upper ˜10 μm of the enamel surface. In this region treatment with T1 leads to a ⁴⁴calcium uptake approx. three and a half times higher than treatment with C2 and approx. five times higher than treatment with C1. C2 has a ⁴⁴calcium uptake ˜1.5 times higher than treatment with C1. In order to assess any statistically significant differences in ⁴⁴calcium uptake between treatment groups, a Student “T” Test has been carried out. All differences in calcium uptake observed with statistically significant.

Conclusion

The greater calcium uptake value observed for the test dentifrice (T1) relative to the comparator formulations (C1 and C2) is indicative of enhanced remineralization of the tooth enamel surface for the test dentifrice.

1. A dentifrice composition comprising a carboxylic acid or alkali metal salt thereof wherein the acid is selected from the list consisting of malonic acid, glutaric acid, tartaric acid, lactic acid and mixtures thereof; and a source of free fluoride ions, and wherein the composition has a slurry pH in the range from greater than 5.0 to less than 6.5.

2. A dentifrice composition according to claim 1 wherein the alkali metal salt is the sodium salt of the said carboxylic acid.

3. A composition according to claim 2 wherein the alkali metal salt is sodium lactate acid present in an amount of 0.5% to 5.0% by weight of the total composition.

4. A composition according to claim 1 wherein the source of free fluoride ions is an alkali metal fluoride.

5. A composition according to claim 4 wherein the alkali metal fluoride is sodium fluoride present in an amount of 0.05% to 0.5% by weight of the composition.

6. A composition according to claim 1 wherein the composition has a slurry pH in the range from 5.4 to 6.3.

7. A composition according to claim 1 wherein the composition comprises a pH modifying agent.

8. A composition according to claim 7 wherein the pH modifying agent is sodium hydroxide.

9. A composition according to claim 1 wherein the composition comprises a copolymer of methyl vinyl ether (MVE) with maleic anhydride or acid.

10. A composition according to claim 9 wherein the copolymer is a copolymer of MVE with maleic acid.

11. A composition according to claim 10 wherein the copolymer is a 1:1 copolymer of MVE with maleic acid.

12. A composition according to claim 9 wherein the copolymer has a molecular weight in the range 100,000 to 2,000,000.

13. A composition according to claim 9 wherein the copolymer is used in an amount from 0.05% to 2% by weight of the composition.

14. A composition according to claim 1 further comprising a desensitising agent.

15. A method of protecting teeth against dental erosion, comprising utilizing the composition according to claim 1.

16. A method of protecting teeth against dental caries, comprising utilizing the composition according to claim 1.
